(19) United States
(12) Patent Application Publication
(10) Pub. No.: US 2026/0217632 A1
(43) Pub. Date: Jul. 30, 2026

(54) IMPROVED ENERGY EFFICIENCY OF METHANOL PRODUCTION FROM CO2

(71) Applicant: CRI, HF, Kopavogur (IS)

(72) Inventors: Pétur Már GÍSLASON, Kopavogur (IS); Viktor STEFANIV, Kopavogur (IS); Armen Eduardo KODJAIAN, Buenos Aires (AR)

(21) Appl. No.: 19/419,661

(22) Filed: Dec. 15, 2025

Related U.S. Application Data
(60) Provisional application No. 63/750,095, filed on Jan. 27, 2025.

(51) Int. Cl. C07C 29/151; B01J 12/00; B01J 19/24; C07C 29/152; C07C 29/78

(52) U.S. Cl. CPC C07C 29/1516; B01J 12/007; B01J 19/2435; C07C 29/152; C07C 29/78

(57) ABSTRACT

Described are improvements that increase the energy efficiency within a chemical synthesis and/or chemical separations plant. Implementations of the present disclosure can be in the form of hot standby operation modes, high-temperature heat pump systems, and additional efficient heat supply systems. The present disclosure provides various embodiments and configurations that can be implemented depending on the needs and challenges of a specific chemical synthesis and/or chemical separations plant.

### CROSS-REFERENCE TO RELATED APPLICATIONS

The present application claims the benefit of U.S. Application No. 63/750,095 (21272.32p.1), filed Jan. 27, 2025, which is herein incorporated by reference in its entirety.

### BACKGROUND

### Technical Field

The implementations of the present invention address the energy efficiency of standby and operation procedures of a chemical synthesis and/or chemical separations plant, e.g., an Emissions-to-Liquid (ETL) methanol synthesis plant.

### Background and Relevant Art

ETL methanol synthesis plants represent an important technological effort to convert carbon emissions and hydrogen gas into valuable chemical products like methanol. ETL plants may rely on a feedstock derived from carbon-containing emissions (e.g., CO₂) and hydrogen to produce methanol via catalytic reactions. Due to the very nature of the feedstock utilized in these ETL plants, inconsistency of supply may pose a significant challenge. ETL plants may rely on variable sources of emissions and/or hydrogen, which can lead to fluctuations in the composition of the syngas feed, as well as fluctuations in the volumetric flow rate of the syngas feed. Such inconsistencies may result in suboptimal reaction conditions, lower methanol yields, and potential catalyst degradation over time. Additionally, some ETL systems may integrate with renewable energy sources, which can introduce inconsistent power input, further complicating process control.

General operational running concerns may add to the overall complexity of ensuring efficient production of such a plant. Achieving the precise balance of temperature, pressure, and reactant flow rates may be important for efficient methanol production. During start-up, these conditions may be difficult to stabilize, leading to inefficiencies and delays in reaching full design production capacity. Heat management may also pose challenges, as methanol synthesis is exothermic, generating excess heat that may need to be carefully controlled to prevent damage to catalysts and equipment. Similarly, the heat transfer mediums themselves may contribute to instability in plant operations when the desired heat transfer capacities cannot be met or are met only periodically. Conventional systems may struggle to adapt to these changing conditions, requiring frequent adjustments and manual intervention, which can reduce efficiency and increase operating costs.

The implementation of the present disclosure addresses one or more, if not all, of the above-described challenges in this field.

### BRIEF SUMMARY

Implementations of the present invention comprise systems and methods configured for improving the overall energy efficiency of a chemical synthesis and/or chemical separations plant through the use of hot standby systems (capable of providing a hot standby mode) and the configuration of a plant's heat integration systems. In particular, implementations of the present invention can comprise a standby loop configuration and/or a catch pot bypass loop configuration. Other described configurations can include at least one high-temperature heat pump, a flash tank, and/or an electric reboiler. The various features may be combined together in any desired combination. Such plants may selectively implement various features to improve operation and energy efficiency, to reach cost and efficiency-based metrics, and/or to better deal with variability of power and variability of feedstock reactant materials and their flow rates.

The terms “plant” and system may be used interchangeably herein.

According to an embodiment, a chemical synthesis hot standby system may comprise a feed line carrying a feed, and a reactor configured for receiving the feed, converting the feed through reaction over a catalyst, so as to produce a product loop gas that is output downstream from the reactor. Such a system may include a heater operatively connected to the feed line and positioned upstream from the reactor and a hot standby loop configured to selectively alter a flow path of the product loop gas when a flow of feed is below a minimum threshold, the hot standby loop putting the reactor in a hot standby mode when selectively activated. The system may also include a product loop gas cooler, downstream from the reactor and downstream from the hot standby loop, the product loop gas cooler being configured for removing heat from the product loop gas when the hot standby loop is not selectively activated and a condenser, downstream from the reactor and downstream from the hot standby loop, wherein latent heat is removed from the product loop gas so as to form a condensed product loop liquid. A catch pot may also be provided, located downstream from the condenser, wherein the condensed product loop liquid is caught and at least partially retained in the catch pot, and wherein any uncondensed product loop gas is removed from the catch pot.

A hot standby system for use in a chemical synthesis plant may comprise a feed line carrying a feed, a reactor configured for receiving the feed, the reactor converting the feed through reaction over a catalyst, so as to produce a product loop gas (e.g., a product stream) that is output downstream from the reactor. The system may also include a heater operatively connected to the feed line and positioned upstream from the reactor, and a product loop gas cooler, downstream from the reactor. The product loop gas cooler may be configured for removing heat from the product loop gas. Such a system may also include a condenser downstream from the reactor and downstream from the hot standby loop. The condenser may be configured to remove latent heat from the product loop gas so as to form a condensed product loop liquid. The system may also include a catch pot bypass configured to selectively alter a flow path of the product loop gas when a flow of feed is below a minimum threshold, the catch pot bypass loop putting the reactor in a hot standby mode when selectively activated. A catch pot may also be included, downstream from the condenser, wherein during a normal operation mode when the flow of feed is above the minimum threshold, the condensed product loop liquid is caught and at least partially retained in the catch pot, and wherein any uncondensed product loop gas is removed from the catch pot. Further, when the reactor is in the hot standby mode, and the catch pot bypass is activated, the catch pot is bypassed.

A high-efficiency steam generation and heat transfer system may include a boiler configured for generating a first steam flow, a flash tank configured for generating a second steam flow from a high-pressure condensate, and an at least one high-temperature heat pump fluidly connected to both the flash tank and the boiler. The at least one high-temperature heat pump may transfer heat from an outside medium to the high-efficiency steam generation and heat transfer system. Such a system may include at least one pump for facilitating the movement of both the first and second steam flows generated by the flash tank and the boiler, and at least one heat exchanger configured to provide heat transfer between the high-efficiency steam generation and heat transfer system and an outside medium. One or more steam line lengths may be included to fluidly connect the boiler, the flash tank, the first high-temperature heat pump, the at least one pump, and the at least one heat exchanger.

A method of operating a chemical synthesis plant in a hot standby mode, may comprise reducing a feed flow rate and selectively actuating at least one valve. The selective actuation of the at least one valve may direct a product loop gas from a standard flow path to a standby flow path. The standby flow path may be a closed loop. The method may include circulating the product loop gas within the standby flow path for a duration. This duration may be defined by the time it takes until the reduced feed flow rate increases by a given threshold value.

A chemical synthesis hot standby system may comprise a feed line carrying a feed. The feed line may include a main feed line and a top feed line. The system may also include a reactor configured for receiving the feed line, and the reactor may convert the feed by reaction over a catalyst so as to produce a product loop gas that is output into a loop gas output. Such a system may include a heater operatively connected to the top feed line and positioned upstream of the reactor and an interchanger operatively connected to the main feed line and a heating medium, the interchanger being configured to transfer heat between the feed in the main feed line and the heating medium. The system may also include a standby loop. This standby loop may include a standby loop line, wherein the product loop gas can flow, the standby loop line being fluidly connected to the loop gas output downstream from the reactor. The standby loop may include a first valve operatively connected to the loop gas output to impede the product loop gas flow inside the loop gas output, and a standby circulator that can selectively facilitate continuous movement of the product loop gas within the standby loop during operation of the standby circulator. The standby loop may also include a second valve operatively connected to the standby loop line upstream of the standby circulator. This second valve may selectively impede the product loop gas from moving through the standby loop. The standby loop may also include a third valve operatively connected to the standby loop line downstream of the standby circulator, and the third valve may selectively impede flow through the standby loop line. Still further, the standby loop may include a fourth valve operatively connected to the top feed line to selectively impede flow through the top feed line. The first, second, third, and fourth valves may selectively alter a flow path of the loop gas. The chemical synthesis hot standby system may also include a product loop gas cooler fluidly connected to the loop gas output, and the product loop gas cooler can be configured for removing heat from the product loop gas. This system may also include a condenser downstream from the reactor and downstream from the hot standby loop. The condenser can be configured to remove latent heat from the product loop gas so as to form a condensed product loop liquid. A catch pot can be fluidly connected to the loop gas output downstream from the condenser. The product loop liquid can be caught and at least partially retained, and the remaining uncondensed product loop gas can be removed from the catch pot. The interchanger, the product loop gas cooler, the condenser, and the catch pot can be bypassed when the standby loop is actuated.

A chemical synthesis hot standby system can include a feed line carrying a feed. The feed line can include a main feed and a top feed. The system can include a reactor configured for receiving the feed and for converting the feed using a catalyst in order to form a product loop gas that is output into a loop gas output. The system can also include a heater which can be operatively connected to the top feed and positioned upstream of the reactor, and an interchanger which can be operatively connected to the main feed and a heating medium, configured to transfer heat between the main feed and the heating medium. A product loop gas cooler that can be fluidly connected to the loop gas output can be included, and it may be configured for removing heat from the loop gas output. A condenser can be positioned downstream from the reactor and downstream from the hot standby loop. The condenser can remove latent heat from the product loop gas so as to form a condensed product loop liquid. The system may also include a catch pot fluidly connected downstream from the condenser. Product loop liquid can be caught and at least partially retained, and the remaining uncondensed product loop gas is removed through a catch pot output. The system may also include a catch pot bypass. This catch pot bypass may include a catch pot bypass line wherein the product loop gas can flow, and a first valve which can be operatively connected to the loop gas output that can selectively alter the product loop gas from flowing through the loop gas output to instead flowing through the catch pot bypass. The catch pot bypass may also include a second valve, which can be operatively connected to the catch pot bypass line. The second valve can impede the product loop gas from flowing through the catch pot bypass. The catch pot bypass may also include a third valve, which can be operatively connected to the catch pot output and can be selectively operable for impeding the flow of product loop gas. The condenser and the catch pot can be bypassed when the catch pot bypass is actuated.

In any of the described embodiments, the feed line can comprise a main feed line and a top feed line.

In any of the described embodiments, a heater can be a standby heater and can be positioned on the top feed line.

In any of the described embodiments, an interchanger can be operatively connected to the main feed line and a heating medium, and the interchanger can be configured to transfer heat between the feed in the main feed line and the heating medium.

In any of the described embodiments, a hot standby loop can include a standby loop line, wherein the product loop gas can flow, and the standby loop line can be fluidly connected to a loop gas output and downstream from the reactor. The standby loop can also include a standby circulator that can be configured to facilitate the continuous movement of the product loop gas within the hot standby loop and a first valve, which can be operatively connected to the standby loop line upstream of the standby circulator, and configured to selectively impede the product loop gas from moving through the hot standby loop. The standby loop can include a second valve, which can be operatively connected to the top feed line to selectively impede flow through the top feed line. The first and second valves can operate to alter the flow path of the product loop gas. opening the first valve, closing the second valve, and turning on the standby circulator can cause the product loop gas to flow circularly through the reactor and the hot standby loop, keeping the reactor in the hot standby mode until the flow of feed surpasses the minimum threshold. Closing the first valve and opening the second valve can return the reactor to a normal operating mode.

In any of the described embodiments, a hot standby loop can comprise a standby loop line, wherein the product loop gas can flow, and the standby loop line can be fluidly connected to a loop gas output and downstream from the reactor. The hot standby loop can include a first valve, which can be operatively connected to the loop gas output to impede the product loop gas flow inside the loop gas output, and a standby circulator, which can be configured to facilitate the continuous movement of the product loop gas within the hot standby loop. The hot standby loop can include a second valve, which can be operatively connected to the standby loop line upstream of the standby circulator and can be configured to selectively impede the product loop gas from moving through the hot standby loop. A third valve can also be operatively connected to the standby loop line downstream from the standby circulator. This third valve can selectively impede flow through the standby loop line. A fourth valve can be operatively connected to the top feed line to selectively impede flow through the top feed line. The first, second, third, and fourth valves operate to alter a flow path of the product loop gas, wherein closing of the first valve, opening of the second valve, opening of the third valve, and closing of the fourth valve causes the product loop gas to flow circularly through the reactor and the hot standby loop, keeping the reactor in the hot standby mode until the flow of feed surpasses the minimum threshold, wherein opening the first valve and closing the second valve returns the reactor to a normal operating mode.

In any of the described embodiments, the feed can be syngas comprising hydrogen and carbon dioxide.

In any of the described embodiments, a catch pot bypass can include a catch pot bypass line wherein product loop gas can flow, a first valve that can selectively allow the product loop gas to flow through the catch pot bypass, and a second valve which can operatively be connected to the catch pot bypass line. The second valve can impede the product loop gas from flowing through the catch pot bypass. The catch pot bypass can include a third valve operatively connected to the catch pot and selectively operable for impeding the flow of product loop gas, wherein closing of the first valve, opening of the second valve, and closing of the third valve can cause the product loop gas to bypass the catch pot.

In any of the described embodiments, a catch pot bypass can include a catch pot bypass line wherein product loop gas can flow, a first valve that can selectively allow the product loop gas to flow through the catch pot bypass, and a second valve which can be operatively connected to the catch pot bypass line. The second valve can impede the product loop gas from flowing through the catch pot bypass. Closing of the first valve and opening of the second valve cause the product loop gas to bypass the catch pot.

In any of the described embodiments, an interchanger can be operatively connected to the main feed line and a heating medium. The interchanger can be configured to transfer heat between the main feed and the heating medium.

In any of the described embodiments, a second high-temperature heat pump can be included and configured to transfer heat from a second outside medium to the high-efficiency steam generation and heat transfer system.

In any of the described embodiments, a boiler can be electric.

In any of the described embodiments, heating the product loop gas can be done with an electric heater within the standby flow path.

In any of the described embodiments, product loop gas can be circulated through a fluidly connected reactor in a standby flow path. The product loop gas can maintain the reactor within a specified temperature range to prevent a cold shutdown of such a reactor.

In any of the described embodiments, one or more valves can be selectively actuated to alter the standard flow path, which may form the standby flow path.

In any of the described embodiments, a reduced feed flow rate threshold value is less than or equal to about 30% of a working flow rate associated with the standard flow path.

In any of the described embodiments, the chemical synthesis plant can comprise a methanol synthesis plant.

In any of the described embodiments, a method can include selectively actuating at least one valve once the reduced feed flow rate increases by the given threshold value. The actuation of the at least one valve can cause the product loop gas from the standby flow path to the standard flow path.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims, or may be learned by the practice of such exemplary implementations as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1A schematically illustrates at least a portion of an ETL methanol synthesis and separations plant wherein a high-temperature heat pump heat integration loop of the present disclosure is implemented;

FIG. 1A-1 schematically illustrates an embodiment of the ETL plant shown in FIG. 1A, having one or more reboilers;

FIG. 1B schematically illustrates another embodiment of a portion of an ETL methanol synthesis and separations plant where a high-temperature heat pump heat integration loop of the present disclosure is implemented, and wherein no air coolers are utilized;

FIG. 1B-1 schematically illustrates a variation in the portion of an ETL methanol synthesis and separations plant shown in FIG. 1B, having one or more reboilers;

FIG. 1B-2 schematically illustrates a variation in the portion of an ETL methanol synthesis and separations plant shown in FIG. 1B, having alternative or additional heat loops;

FIG. 1B-3 schematically illustrates a variation in the portion of an ETL methanol synthesis and separations plant shown in FIG. 1B, having alternative or additional heat loops;

FIG. 1C-1 illustrates another embodiment of a portion of an ETL methanol synthesis and separations plant shown in FIG. 1B, having no flash tank, including one or more reboilers;

FIG. 1C-2 illustrates another embodiment of a portion of an ETL methanol synthesis and separations plant shown in FIG. 1C-1, having an electric boiler;

FIG. 1C-3 illustrates another embodiment of a portion of an ETL methanol synthesis and separations plant shown in FIG. 1C-1;

FIG. 2A schematically illustrates an exemplary embodiment of a hot standby system of the present disclosure, wherein an electric standby heater and air cooler are utilized;

FIG. 2B schematically illustrates another embodiment of a hot standby system of the present disclosure, wherein an electric standby heater and heat transfer mechanism are utilized;

FIG. 2C schematically illustrates another embodiment of a hot standby system of the present disclosure, wherein an electric standby heater is utilized;

FIG. 3A schematically illustrates a heat supply system of an ETL methanol synthesis plant where a reboiler and boiler are configured to generate and utilize steam; and

FIG. 3B schematically illustrates another heat supply system for an ETL methanol synthesis plant of the present disclosure where an electric reboiler is used.

### DETAILED DESCRIPTION

The implementations of the present disclosure extend to Implementations of the present invention comprise systems and methods configured for improving the overall energy efficiency of a chemical synthesis and/or chemical separations plant through the use of hot standby systems (capable of providing a hot standby mode) and the configuration of a plant's heat integration systems. In particular, implementations of the present invention can comprise a standby loop configuration and/or a catch pot bypass loop configuration. Other described configurations can include at least one high-temperature heat pump, a flash tank, and/or an electric reboiler. The various features may be combined together in any desired combination. Such plants may selectively implement various features to improve operation and energy efficiency, to reach cost and efficiency-based metrics, and/or to better deal with variability of power and variability of feedstock reactant materials and their flow rates.

The presently disclosed ETL methanol synthesis plants convert a syngas feedstock or carbon-containing emissions, such as CO₂ or other greenhouse gases, into methanol or other value-added products through a chemical process, often involving hydrogen and catalysts. While this technology is appealing due to its production of methanol, which can be used as a fuel or chemical feedstock, the operation of an ETL plant presents several unique challenges, primarily due to the complex chemical processes, the integration of various subsystems, and the need to maintain precise operating conditions, each of which can be exacerbated by the fact that flow rates of feedstock reactants (e.g., CO2 and H2) may vary. Similarly, the compositional characteristics of the feedstock stream may also vary over time. Such difficulties may make it advantageous to provide a hot standby mode within such a plant, when a minimum threshold flow rate of a needed feedstock composition is not readily available.

### Section 1—High-Temperature Heat Pump Heat Integration Loop

Methanol synthesis is an exothermic process. When produced from carbon monoxide (CO), CO+2H₂→CH₃OH, the reaction has a delta H (ΔH) or heat of reaction value of about −90.7 kJ/mol (negative, indicating exothermicity). When produced from carbon dioxide (CO₂), CO₂+3H₂→CH₃OH+H₂O, the reaction has a ΔH of approximately −49.5 kJ/mol (negative, indicating exothermicity). In either case or a combination of such reactions, the release of energy or heat during methanol production needs to be managed to ensure that damage to the catalyst, reactor, product, or feed does not occur. Furthermore, various steps within the overall ETL plant require either the introduction or removal of heat. Because of this, an ETL plant must account for that heat generated in the system so that it does not cause adverse thermal stress and cycling on equipment, which can lead to operational inefficiencies or mechanical failure. Conventional ETL methanol synthesis plants often involve fine-tuning heat exchange and cooling systems to ensure they function properly in dynamic operational conditions. An operator can implement a given embodiment of the present disclosure into a new or existing ETL plant or into other exothermic chemical synthesis plants or processes to ensure that heat is transferred effectively and efficiently. Such embodiments may be particularly applicable to plants that seek to reduce or eliminate the use of fossil fuel sources of power or heat, e.g., instead opting to use renewable and other green energy sources.

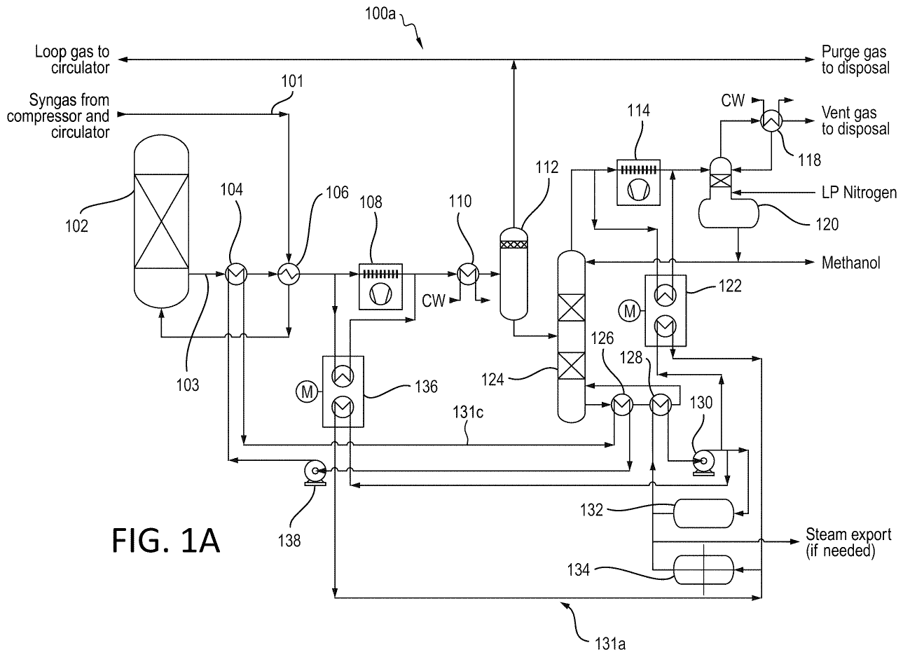

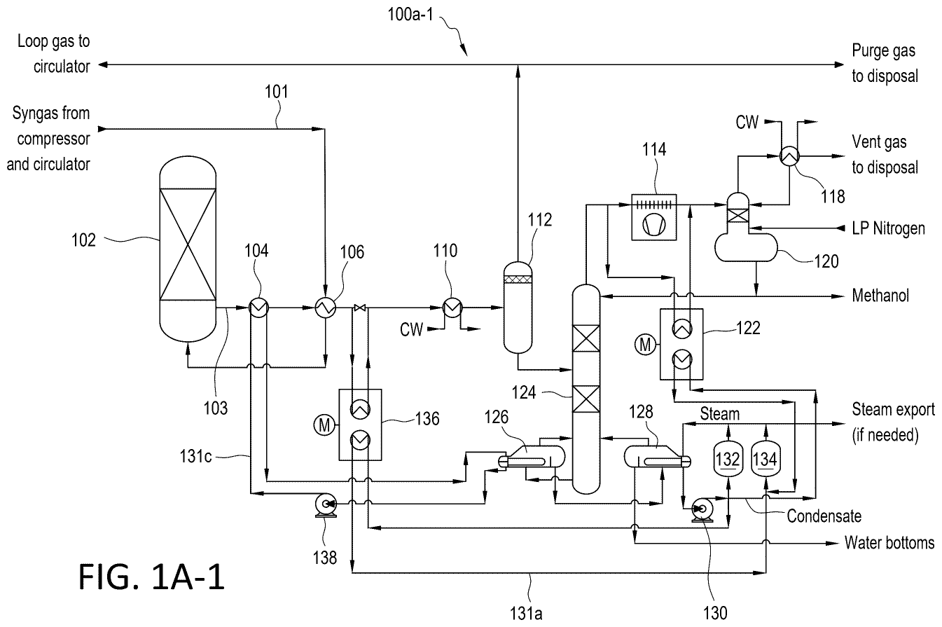

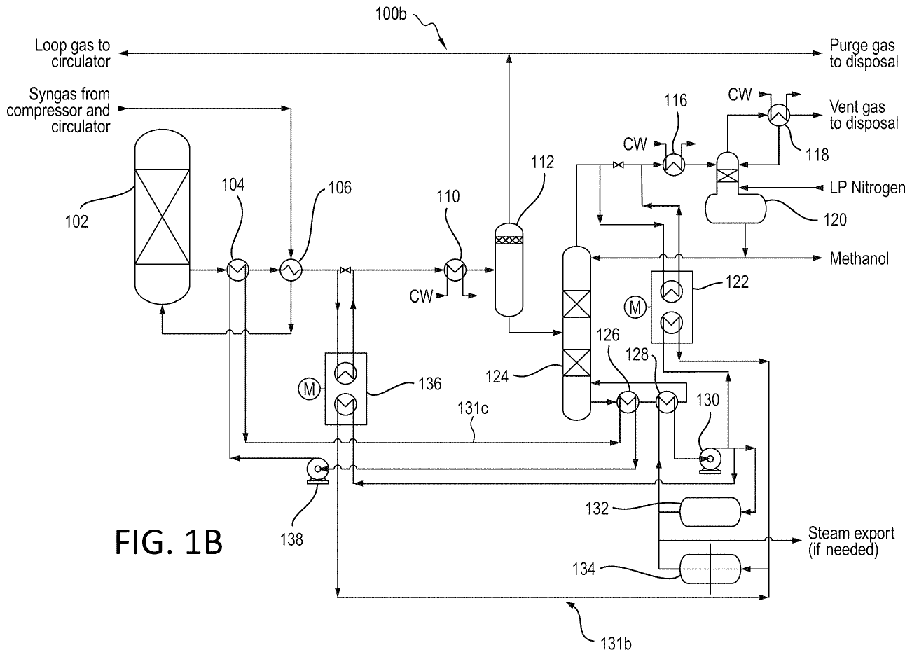

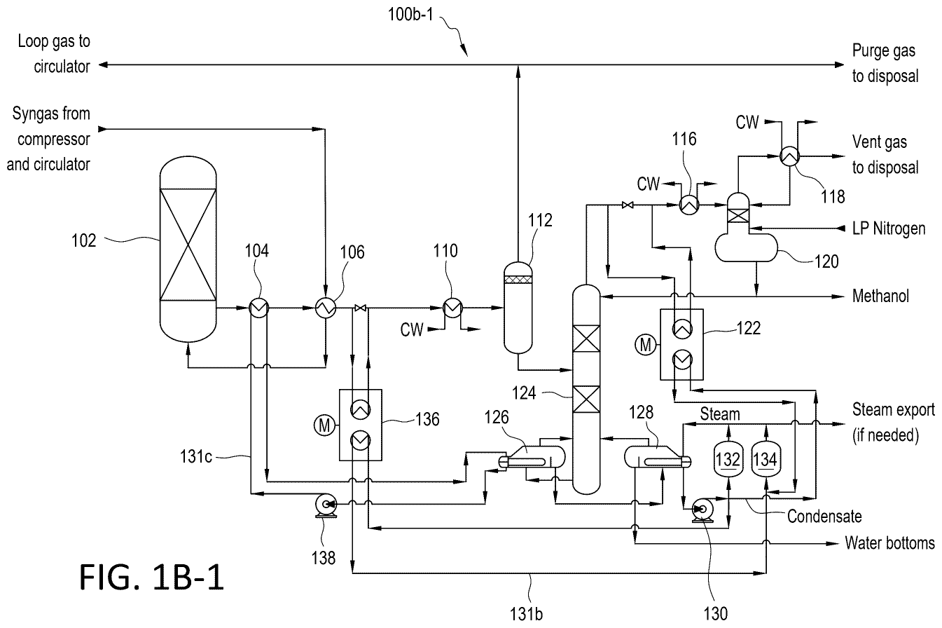

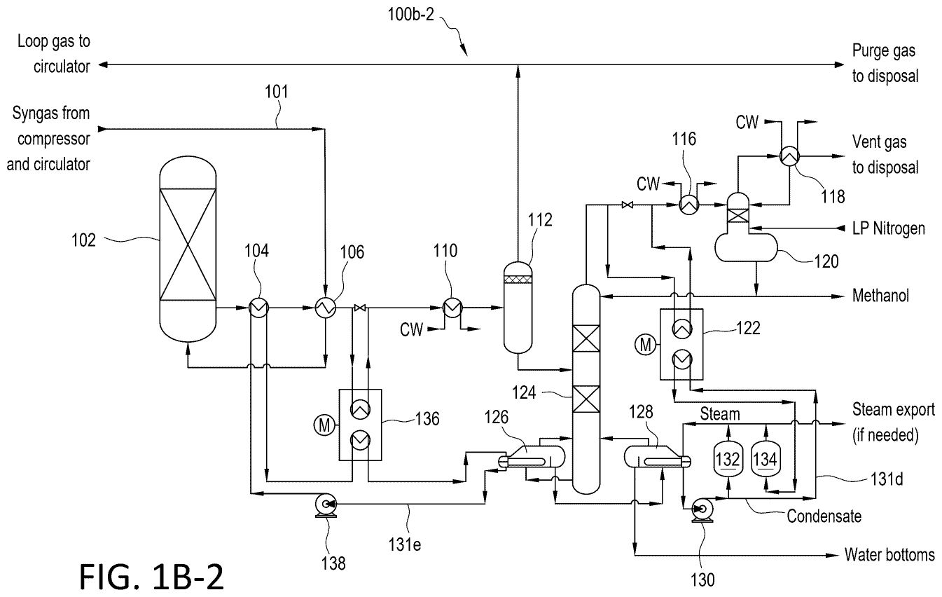

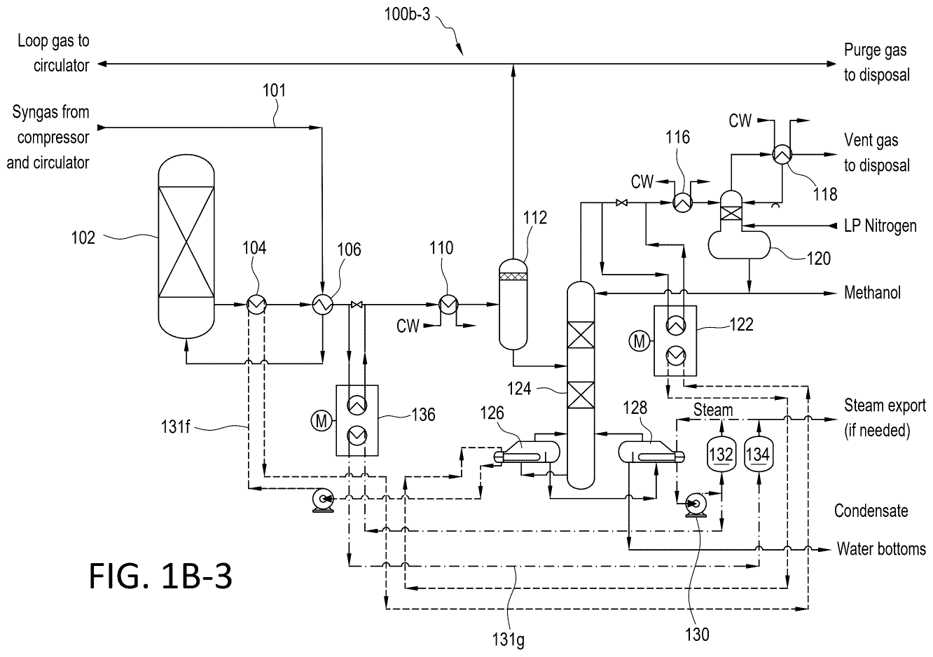

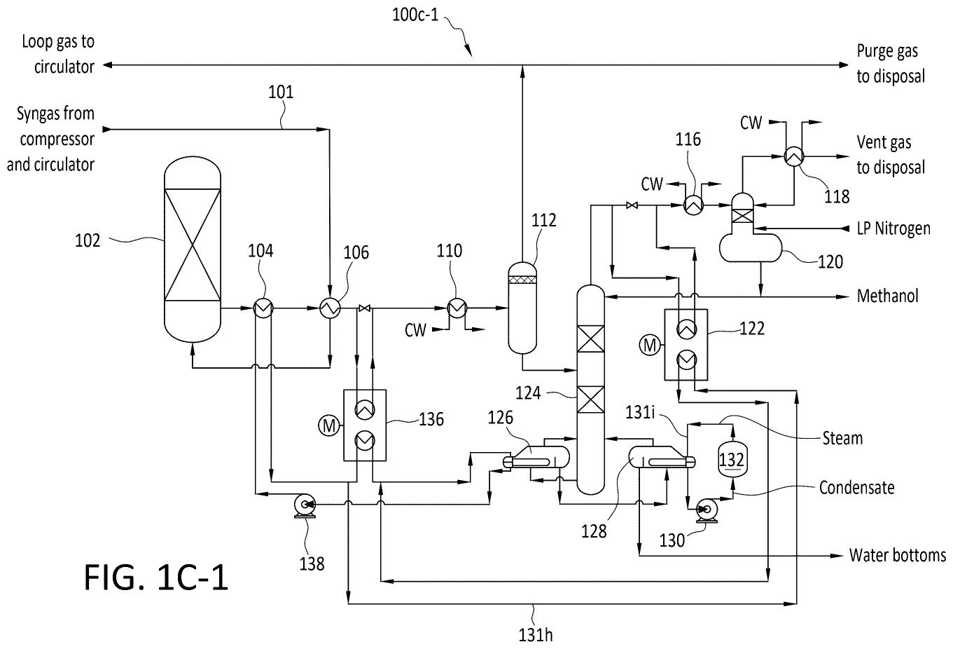

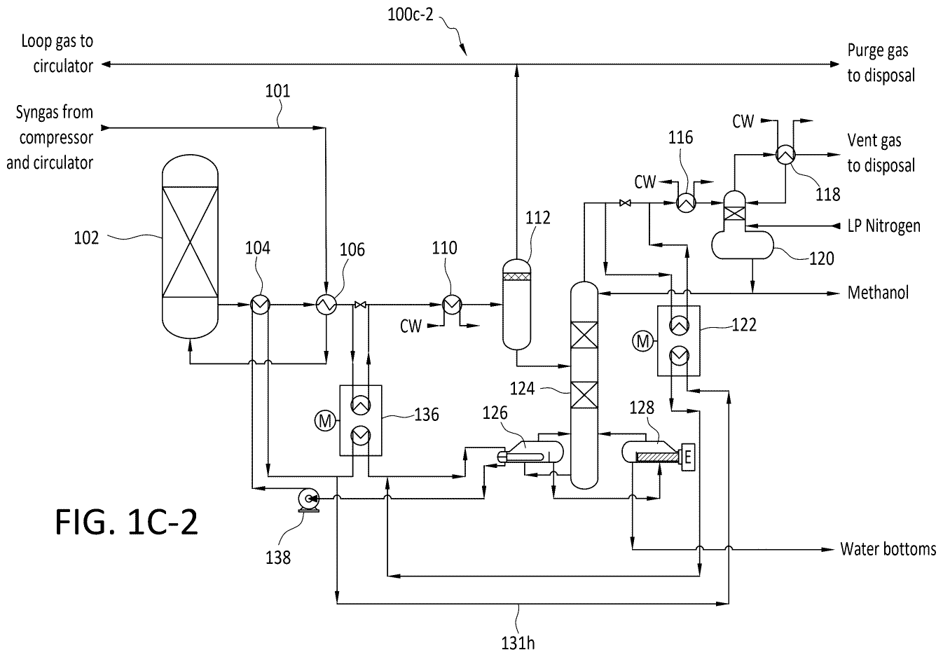

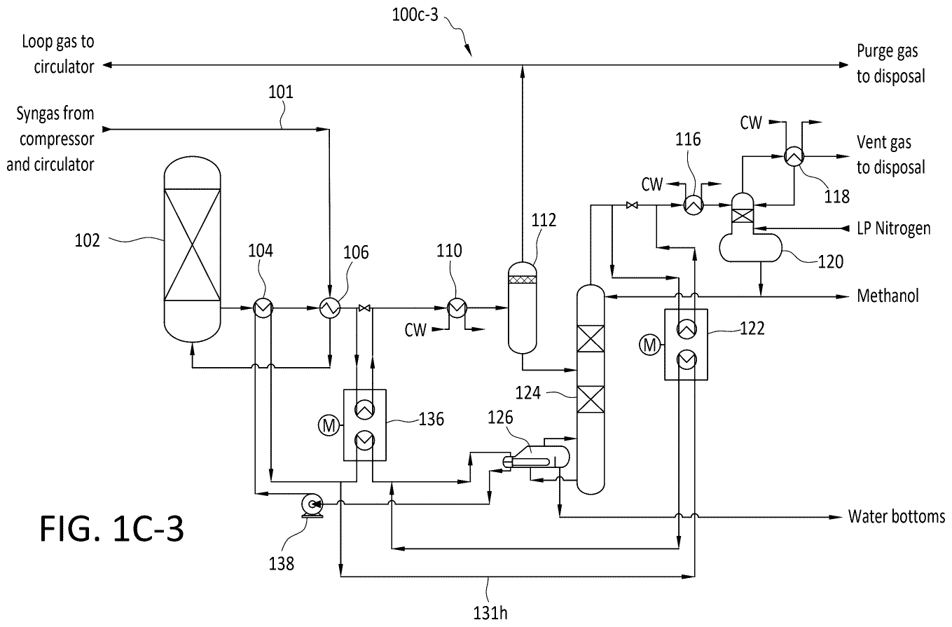

FIGS. 1A and 1B schematically illustrate at least a portion of an ETL methanol synthesis plant or system 100a that includes a high-temperature heat pump heat integration loop. Such a plant or system 100a, including the high-temperature heat pump integration loop, can be organized and implemented in various ways, depending on the plant in which it is installed. It is understood that any such system 100a, including the high-temperature heat pump heat integration loop, can include the same equipment as illustrated in FIG. 1A or 1B. Still, in another such implementation, such a system can include additional or substantially different pieces of equipment.

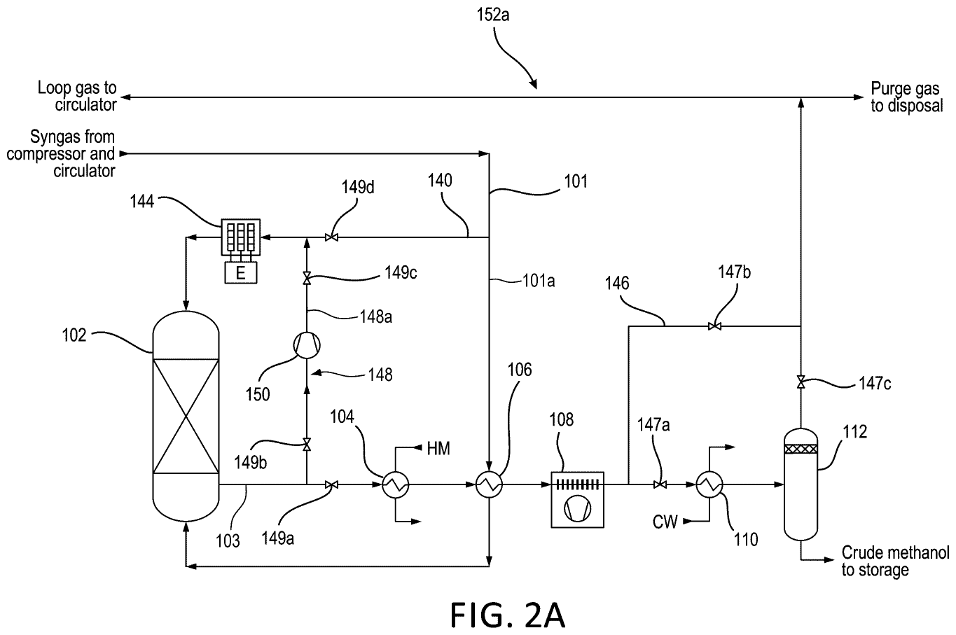

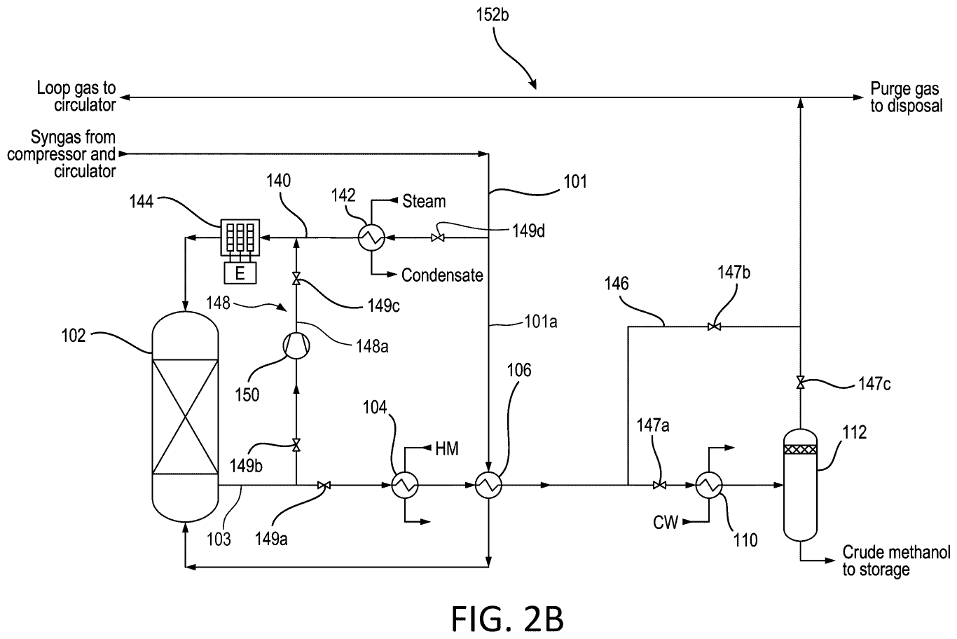

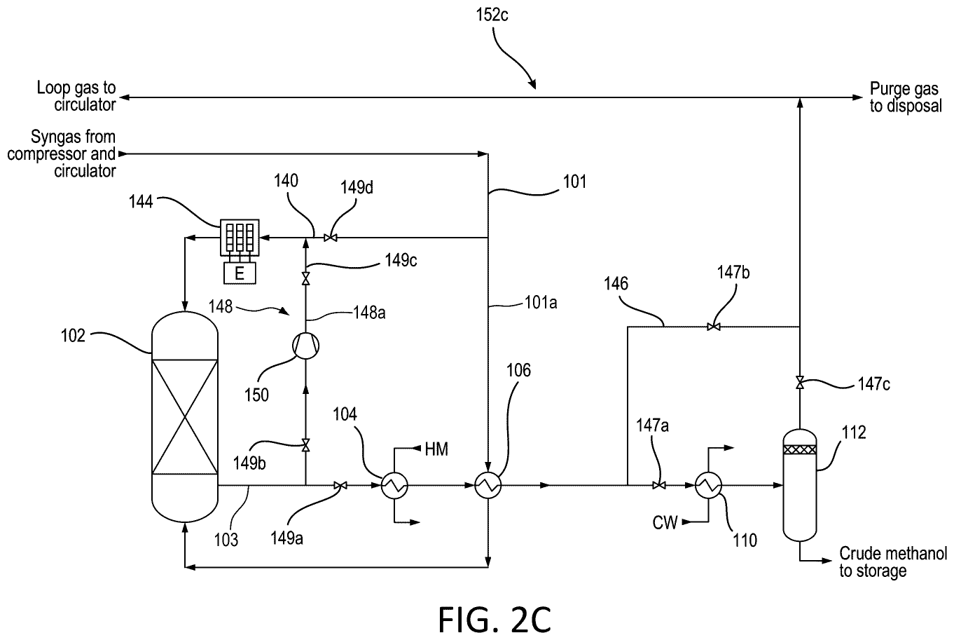

Feedstock line 101 provides for a flow of a mixed syngas stream of H₂, CO, and CO₂. In an embodiment, CO may or may not be present (e.g., the stream may be substantially void of CO, or the carbon oxide component may predominantly comprise CO₂). Feedstock line 101 can be heated by heat exchanger 106, where the heat from the outlet gas (e.g., loop gas) of reactor 102 is used as a heat transfer medium. Feedstock line 101 can also be split so that top feed line 140 (as seen in FIG. 2A) is fed into the top of reactor 102, where the feedstock is heated with a heating medium separate from the loop gas heat produced from the reactor. Pressurized flow and movement of this stream can come from a feed compressor and circulator, e.g., located upstream from feedstock line 101, as shown. Reactor 102 can house a catalyst bed wherein the catalyst promotes a methanol synthesis reaction as the feedstock is passed through the catalyst bed. By way of example, reactor 102 can output the loop gas (e.g., at 103 in FIG. 1A) at 230° C., 220° C., or at least 210° C. (e.g., from 250° C. to 210° C.). The loop gas output 103 can flow through heat exchangers 104 and 106 to lower the loop gas temperature from, e.g., about 240-250° C. down to about 100-120° C. Heat exchanger 106 can increase the feedstock temperature to about 180-220° C. from its initial temperature of 40-80° C. (e.g., in feedstock line 101).

Loop gas is then sent downstream to air cooler 108, where air cooler 108 removes heat from the loop gas, e.g., to the minimum temperature achievable by air cooling. By way of example, air cooler 108 can cool the loop gas from a temperature of 100-120° C. down to a temperature of 50-60° C. In at least one embodiment, a given plant may not include such an air cooler 108. Air cooler 108 aids in reducing the demand for cooling water or other cooling medium needed and may also partially condense the loop gas. As shown in FIG. 1A, at least a portion of the loop gas can be routed through high-temperature heat pump 136, e.g., bypassing the air cooler 108. A high-temperature heat pump is understood to be configured to transfer thermal energy from one medium to another (e.g., pumping heat “uphill” from the lower temperature medium to the higher temperature medium). Further, a high-temperature heat pump can allow for a lower-quality medium, e.g., loop gas, to be used for heat transfer and still be effective. Heat is taken from a low-quality or temperature medium by cooling it down, wherein the temperature of the working medium is further increased inside the heat pump via a reverse sterling cycle. This higher-temperature heat is transferred to a high-temperature heat sink, heating another medium, such as an outside medium. As used herein, an outside medium can be any system, plant, fluid, loop, etc., where heat can be transferred. For example, an outside medium can include a different loop within the same system, so long as it is not fluidically connected. As illustrated then, high-temperature heat pump 136 can work alone or in tandem with air cooler 108 to transfer heat from the loop gas and impart that heat into steam (and/or condensate) within a steam loop, raising the overall grade of the steam interfaced on the other side of high-temperature heat pump 136 (e.g., for use in providing heat to the bottoms portion in a separation column). As used herein, steam or steam flow may also refer to the condensate within a steam generation loop. In another implementation, the transferred heat from the loop gas can be imparted into another medium through the same high-temperature heat pump 136. In at least one embodiment, a high-temperature heat pump heat integration loop may not employ both high-temperature heat pumps 136 and 122 as shown in FIG. 1A. For example, where high-temperature heat pump 122 is present, heat pump 136 may be omitted. A plant implementing both high-temperature heat pumps 122 and 136 would advantageously increase the overall coefficient of performance (COP) as a plant can pump only the top part of the heat grade range from each of the heat pumps. Of course, such increased COP comes at additional capital expense. A plant can export any additional steam not needed, where both high-temperature heat pumps 122 and 136 are used. Additionally or alternatively, a single high-temperature heat pump may include a plurality of smaller high-temperature heat pumps connected in parallel.

It should be noted that in any of the described embodiments, any number of high-temperature heat pumps can be used to transfer heat between any number of the streams described herein, including but not limited to (i) the loop gas leaving an interchanger and (ii) the streams leaving or entering a distillation column(s). Thus, though the illustrated embodiment shows locations and positions for the high-temperature heat pumps 122 and 136, these high-temperature heat pumps (or additional or alternative high-temperature heat pumps) may be positioned at various other location within their associated plants or systems.

Condenser 110 can remove latent heat from the loop gas, forming condensed methanol. The methanol and remaining loop gas can then flow into catch pot 112. Catch pot 112 can be configured to pool (catch and retain) methanol and release any remaining uncondensed loop gas, which may be recycled or otherwise purged. The loop gas, if recycled, can be reheated before, during, or after mixing with the feedstock. By way of example, the loop gas may predominantly comprise unreacted CO₂, CO and/or H₂, along with any other byproducts, after the methanol product has been removed. Condenser 110 can be designed to cool the loop gas and resulting methanol to ensure that the pooling condensed methanol is not evaporated by the entering high-temperature methanol or loop gas. Condenser 110 can output loop gas and resulting methanol in the temperature range of 30-50° C.

The resulting crude methanol product can then be fed into separation column 124. While not limiting, column 124 can be a distillation column, a fractioning tray column, and/or a packed column such that the crude methanol feed undergoes further separation or purification. Column 124 can operate under relatively low pressures, such as 0 to 5 bar (gauge), or at higher pressures of 5 to 50 bar or 50 to 100 bar (gauge). Air cooler 114 can be installed so as to receive the top fraction from column 124 to remove heat from the top fraction before the top fraction flow enters a stripper unit 120. Stripper unit 120 can use a stream of a nonvolatile substance, e.g., low-pressure nitrogen, to remove volatile impurities that may be present in the top fraction stream. The purified top fraction can then be collected for storage or use. The plant can also use heat exchanger 118 to cool the top fraction, with recycle of a portion thereof as shown, to improve the yield of stripper unit 120. In at least one embodiment, heat exchanger 118 can be used as a condenser to remove latent heat (where the condensate fraction is recycled to stripper unit 120, as shown).

High-temperature heat pump 122 can be a heat transfer mechanism between the top fraction exiting column 124 and another working medium within the plant (e.g., bypassing air cooler 114). As illustrated, high-temperature heat pump 122 transfers heat from the top fraction output from column 124 to a steam generation loop or heat loop 131a (high-temperature heat pump 136 also transfers heat into steam generation loop or heat loop 131a). As described herein, while the system 100a including high-temperature heat pump heat integration loop is shown as including both high-temperature heat pumps 136 and 122, in another embodiment, one may choose to install either high-temperature heat pump 122 or 136 depending on the plant's needs. One skilled in the art will appreciate that operating both high-temperature heat pumps 122 and 136 will advantageously increase the COP associated with the steam generation loop or heat loop 131a.

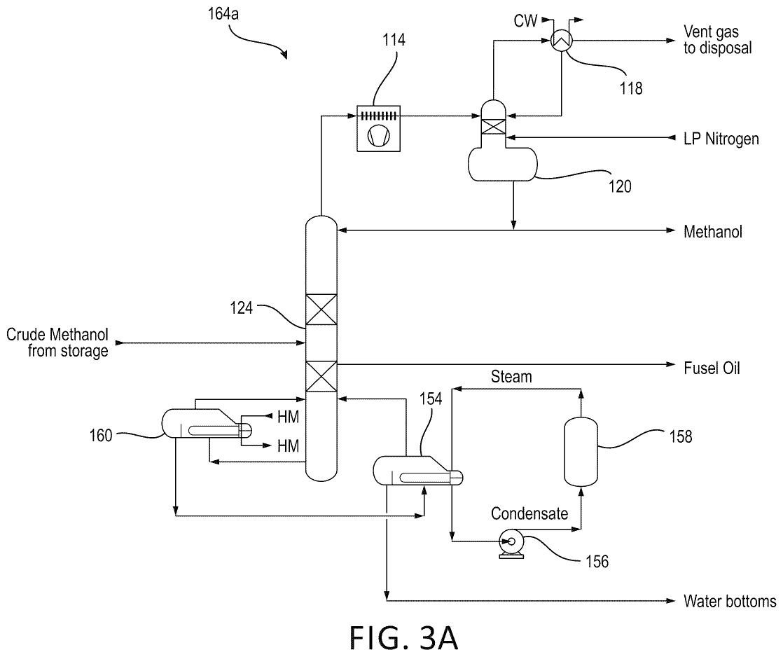

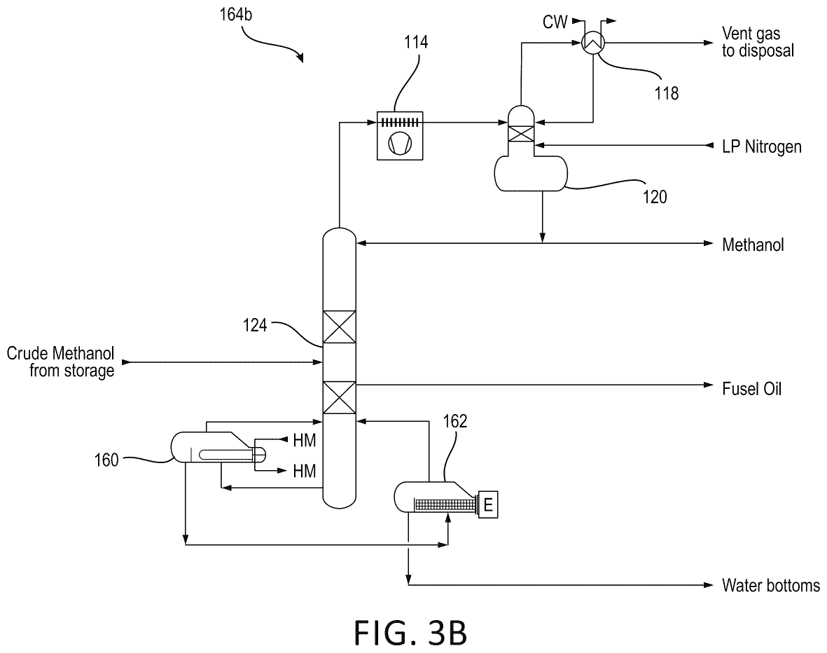

As illustrated, the bottom fraction from column 124 can be circulated through a series of heat exchangers (e.g., 126 and 128). FIG. 1A shows heat exchangers 126 and 128 being used in series to provide heat to the bottom fraction of column 124 (e.g., such heat exchangers may replace or otherwise serve as a reboiler). Heat exchanger 126 and heat exchanger 104 utilize a working medium that can transfer heat from loop gas exiting reactor 102 to the bottom fraction of column 124 within heat loop 131c. Heat loop 131c may be a closed loop, as shown. In an embodiment, this working medium can have a temperature of about 150-200° C. The working medium within heat loop 131c is moved using pump 138. Heat exchanger 128 can be part of steam generation loop or heat loop 131a, thus allowing for the steam generated in steam generation loop or heat loop 131a to increase the temperature of the bottom fraction of column 124, before such bottom fraction is reintroduced into the column 124 as shown. In another embodiment, as seen in FIGS. 3A and 3B, one or more reboilers can be installed in addition to, or replacement of heat exchangers 126 and 128, so that the bottom fraction is heated to form a vapor from the bottom fraction liquid.

Steam generation loop or heat loop 131a can comprise a boiler 132, a flash tank 134, a pump 130, and high-temperature heat pumps 122 and 136. In another embodiment, a plant can use more or fewer components to achieve a desired COP and to ensure that each step in the chemical separation process is performed at the desired temperatures. Steam generation loop or heat loop 131a can be configured to produce saturated or nearly saturated grade steam at a temperature of approximately 120-200° C., such as 120-150° C., or 160-180° C. As used herein, “near” or “nearly saturated” grade steam refers to steam having a temperature not more than 10° C. above its saturation temperature. A plant can utilize either a fuel boiler, an electric boiler, or any other type of boiler at boiler 132. One skilled in the art will appreciate that the addition of flash tank 134 into steam generation loop or heat loop 131a will advantageously improve energy efficiency and operation costs.

In one embodiment, flash tank 134 can allow for the high-pressure condensate within steam generation loop or heat loop 131a to “flash” into low-pressure steam, i.e., allowing for more energy to be effectively used out of the produced steam than if no flash tank was installed. When used in conjunction with the high-temperature heat pumps as shown, this lower-pressure or otherwise lower-quality steam can be stepped up to be an effective heat transfer medium. In another embodiment, flash tank 134 is piped in parallel with boiler 132, e.g., as boiler 132 may only be used in start-up procedures while the heat pumps cannot be used. Flash tank 134 can then maintain the plant's applicable steam requirements during normal operation. A plant can also combine flash tank 134 and boiler 132 into a kettle-type electric boiler/flash drum to achieve the benefits of this embodiment. In at least one embodiment, steam generated in steam generation loop or heat loop 131a can be exported to other processes within the plant or another plant, or to processes outside of the plant. In such a case, steam generation loop or heat loop 131a can produce a single grade or multiple grades of steam and export any excess steam or even used steam that may now be at a lower grade.

FIG. 1A-1 illustrates an embodiment of the ETL plant shown in FIG. 1A (methanol synthesis plant or system 100a-1), wherein the heat exchangers 126 and 128 are represented as reboilers. All features and explanations regarding FIG. 1A are thus applicable to the features of FIG. 1A-1.

FIG. 1B schematically illustrates another embodiment of a portion of an ETL methanol synthesis plant or system 100b, including a high-temperature heat pump heat integration loop. System 100b can comprise substantially the same components described above regarding system 100a. FIG. 1B illustrates an embodiment of a system 100b including a high-temperature heat pump integration loop wherein neither air cooler 108 nor air cooler 114 is present. In place of air cooler 114, heat exchanger 116 is utilized to remove heat from the top fraction exiting from column 124. As in FIG. 1A, a plant can optionally omit either high-temperature heat pump 122 or 136 depending on the heat and efficiency needs of that given plant. In at least one embodiment of the disclosure, high-temperature heat pumps 122 and 136 may refer to a bank or grouping of high-temperature heat pumps, e.g., run in parallel or in series to not only address the heat integration needs of the particular plant or system but to also provide savings associated with utilizing smaller or less expensive high-temperature heat pumps rated for lower individual capacities.

FIG. 1B-1 schematically illustrates a variation in the portion of an ETL methanol synthesis plant or system 100b wherein the heat exchangers 126 and 128 are represented instead as reboilers (i.e., methanol synthesis plant or system 100b-1). As illustrated, heat loop 131c still operably connects heat exchanger 104 with the column 124, but utilizes a reboiler as heat exchanger 126. Heat loop 131b that connects high-temperature heat pump 136 is still operably connected with boiler 132 and flash tank 134. Thus, heat loop 131c can transfer heat between the bottom fraction of column 124 and the loop gas leaving reactor 102.

FIG. 1B-2 schematically illustrates a variation in the portion of an ETL methanol synthesis plant or system 100b wherein heat loops 131b and 131c are replaced with heat loops 131d and 131e (i.e., methanol synthesis plant or system 100b-2). Heat loop 131d operably connects high-temperature heat pump 122 with boiler 132 and flash tank 134, and any steam produced by them. Similarly, heat loop 131d is operably connected to heat exchanger 128, where heat exchanger 128 is a reboiler. Heat loop 131e operable connects heat exchanger 104 with high-temperature heat pump 136 and heat exchanger 126, where heat exchanger 126 is a reboiler. Thus, in this embodiment, high-temperature heat pump 136 is not operably connected within the same heat loop as high-temperature heat pump 122.

FIG. 1B-3 schematically illustrates another variation of the portion of an ETL methanol synthesis plant or system 100b wherein heat loops 131b and 131c have been replaced by heat loops 131f and 131g (i.e., methanol synthesis plant or system 100b-3). Heat loop 131f operably connects high-temperature heat pump 122 with heat exchanger 104 and heat exchanger 126, where heat exchanger 126 is a reboiler. Thus, the top fraction of column 124, the bottom fraction of column 124, and the loop gas leaving reactor 102 are thermally coupled. Heat loop 131g operably connects high-temperature heat pump 136 with heat exchanger 128 (where heat exchanger 128 is a reboiler), boiler 132, and flash tank 134. Thus, heat loop 131g thermally couples steam generated in the boiler and/or flash tank with the bottom fraction of column 124 and the loop gas leaving reactor 102.

FIG. 1C-1 illustrates another embodiment of a portion of an ETL methanol synthesis plant or system similar to methanol synthesis plant or system 100b, wherein there is no flash tank and heat exchangers 126 and 128 are reboilers (i.e., methanol synthesis plant or system 100c-1). In this embodiment, heat loop 131h thermally couples the loop gas leaving the reactor 102 with the bottoms fraction of column 124, the top fraction of column 124. In this embodiment, heat loop 131h thus operably connects together heat exchanger 104, heat exchanger 126 (where it is a reboiler), and both high-temperature heat pumps 122 and 136. Thus, heat loop 131i is a standalone loop that cycles steam from boiler 132 only with the bottom fraction of column 124 that passes through heat exchanger 128 (where it is a reboiler).

FIG. 1C-2 illustrates another embodiment (i.e., methanol synthesis plant or system 100c-2) of a portion of an ETL methanol synthesis plant or system similar to methanol synthesis plant or system 100b, such as that shown in FIG. 1C-1; however, heat loop 131i of FIG. 1C-1 is replaced with an electric reboiler (heat exchanger 128). The use of an electric reboiler can allow for the introduction of the desired heat in a simple and low-component-count solution (e.g., no boiler, pump, or piping).

FIG. 1C-3 illustrates an embodiment (i.e., methanol synthesis plant or system 100c-3) of a portion of an ETL methanol synthesis plant or system similar to methanol synthesis plant or system 100b, such as that shown in FIG. 1C-1; however, no boiler or electric reboiler is used. Rather, the plant or system only utilizes heat loop 131h, which leverages the existing heat within the plant or system to meet the plant or system requirements. As shown, high-temperature heat pumps 122 and 136, heat exchanger 104, and heat exchanger 126 (where it is a reboiler) act together to move heat between the loop gas leaving reactor 102, the bottoms fraction of column 124, and the top fraction of column 124.

Though FIGS. 1A-1C-3 illustrate various and distinct heat loop configurations, any concept within each of the Figures can be shared or implemented within any other of the described embodiments in order to accomplish the goal of providing or removing heat within a chemical synthesis plant.

One skilled in the art will appreciate the overall energy and operational cost savings that the steam generation loops or heat loops 131a, 131b, 131c, 131d, 131e, 131f, 131g, 131h, and 131i of FIGS. 1A-1C-3 may provide within a chemical separations process. When the high-temperature heat pump heat integration loops as described is integrated into such a plant or system 100a or 100b, a plant can use two or more times less electricity compared to a similar ETL plant which relies only on electric heaters to generate steam and does not include the high-temperature heat pump heat integration loops. While the initial capital expense will be higher, the operational costs will be substantially lower. Another benefit associated with such a configuration is that such a plant would have little or no need for importation of additional heat, where steam generation loops or heat loops 131a, 131b, 131c, 131d, 131e, 131f, 131g, 131h, and 131i are implemented into the process. In locations where steam is difficult or impossible to source or where another heat integration medium is of intermittent or in short supply, implementations of the present disclosure allow for the constant running of the plant without the need for any outside heat integration medium. In cases described herein where a plant may intermittently use, and/or fluctuating functioning and flowrates of feedstock and produced product, steam integration loops or heat loops 131a and 131b, 131c, 131d, 131e, 131f, 131g, 131h, and 131i can be independently started (e.g., wherein boiler 132 acts as a start-up boiler) or stopped based on the plant's heat integration needs.

### Section 2—Hot Standby System

The feedstock of reactants of an ETL plant are the lifeblood of the entire process, meaning interruptions, inconsistencies, or incorrect stoichiometric ratios within a feedstock dictate not only the output of the ETL plant but also the ability of an ETL plant to remain active or operate at any given time. Feedstock and ETL methanol synthesis plant reactants may include a mixture of H₂, CO, and CO₂. Often, this feedstock of reactants is sourced from variable rate sources like emissions from industrial processes, biomass gasification, and/or renewable energy-driven electrolysis (e.g., for H₂), meaning their continuous and steady state flow may not be reliable. In addition to fluctuations in flowrate, these feedstocks can fluctuate in composition, particularly in the ratios of hydrogen, carbon monoxide, and carbon dioxide, leading to an imbalance in the syngas mixture desired or required for efficient methanol synthesis. Such variations can disrupt the catalytic process, reducing methanol yield and potentially damaging the catalyst over time. Additionally, inconsistent feedstock can cause undesirable reactor temperature and pressure fluctuations, requiring constant adjustments to maintain optimal operating conditions. This variability complicates process control, increases downtime, and can lead to higher operational costs, as more energy and resources may be needed to stabilize the system and achieve the desired methanol output.

A conventional ETL plant would typically need to be shut down during long periods of significantly reduced feedstock flow, as such synthesis and purification can be a complex process that must be carefully managed to avoid damaging equipment or wasting resources. Implementations of the present disclosure, however, provide a plant or system with an alternative hot standby operational mode wherein the costly and time-intensive process of shutdown and start-up can be avoided by allowing the reactor of an ETL plant to remain warm and otherwise ready for when the inconsistencies or interruptions in desired flowrates end (e.g., between 200° C. to 300° C.). When faced with a lack of feedstock of reactants, operators can gradually reduce the flow of feedstock or reactants into the reactor to protect the catalyst. At the same time, heat management systems, such as cooling circuits and heat exchangers, can gradually be adjusted to safely dissipate and distribute the residual heat generated by the methanol synthesis reaction, avoiding thermal shocks to the system. During shutdown, operators also typically purge the reactor and associated piping of any remaining process gases to prevent contamination or unwanted reactions during idle periods. This can involve operators pumping inert gases, like nitrogen, to flush the system. Such steps are cumbersome and expensive.

If the plant has additional integrated power and steam sources, those systems must also be carefully powered down, especially in plants utilizing variable energy sources like renewable energy sources, to ensure no excess energy load remains in the system. Once feedstock of reactants, or any other limiting requirements such as heat or other energy, are returned to their operational levels, the process needs to be restarted, which involves again the purging of the reactor and system of any inert gas with the flow of the feedstock of reactants and the slow restart of the chemical reaction. A conventional plant may need supplementary heat integration processes (e.g., various additional heaters and the like) while the entire process ramps up to operational conditions.

FIGS. 2A, 2B, and 2C schematically illustrate at least a portion of an ETL plant or system 152a, 152b, 152c wherein implementations of a hot standby system (capable of standby operational modes) are present. Specifically, FIG. 2A schematically illustrates at least a portion of an ETL plant or system 152a wherein feedstock is fed through feedstock line 101. Feedstock line 101 can be split into main feedstock line 101a and top feed line 140 to allow at least a portion of the feedstock to be fed into reactor 102 under varied conditions. FIG. 2A illustrates that the feedstock within top feed line 140 can be heated by standby heater 144, as, while typically, heat exchanger 106, or some other form of heat exchanger, can be used to raise the temperature of the feedstock for introduction into the reactor. If the reactor is not producing enough exothermal heat, heat exchanger 106 would not be able to impart the desired temperature increase to the feedstock. Thus, standby heater 144 can raise the feedstock temperature during reactor operation at low turndown to temperatures needed for the reaction. In some cases, the pre-heated feedstock leaving heat exchanger 106, just before entering the reactor (the bottom of the reactor), can be 180° C. to 220° C., though the temperature can be higher or lower depending on the composition of the feedstock. As additional pre-heating occurs within the reactor, before the reactants reach the top portion of the reactor, standby heater 144 may heat the reactants to a higher temperature than heat exchanger 106. For example, standby heater 144 may heat the reactants to a temperature greater than 120° C., such as from 150 to 300° C., or from 150 to 250° C.

Reactor 102 can house a catalyst bed wherein the catalyst promotes a methanol synthesis reaction as the feedstock and reactants are passed through the catalyst bed. Reactor 102 can be a fixed bed reactor, a fluidized bed reactor, a slurry bubble column reactor, a membrane reactor, or any other type or form of reactor or converter that allows for producing methanol from at least a syngas feed. The catalysts used in reactor 102 can include but are not limited to copper-based catalysts (Cu/ZnO/Al₂O₃), modified copper catalysts (with chromium or manganese), cobalt and molybdenum catalysts, zirconium-based catalysts (Cu/ZrO₂), ruthenium-based catalysts (Ru-based), or any other experimental and alternative catalysts that would aid in the production, reaction stability, or conditions within reactor 102.

Reactor 102 can operate at temperatures of 200° C. to 300° C. Further, reactor 102 can output a loop gas at a temperature of at least 230° C., at least 220° C., or at least 210° C. By way of example, the exiting loop gas exiting reactor 102 can be output from 210° C. to 230° C. The loop gas output 103 can then carry the loop gas through heat exchanger 104, followed by heat exchanger 106 to lower the loop gas temperature from 190-230° C. to 100-120° C. and subsequently increase the feedstock temperature from 40-80° C. to 180-220° C. It will be appreciated that while methanol synthesis is primarily described, the embodiments and configurations described herein may be implemented in other types of chemical synthesis systems. Of course, the actual temperatures at various points within the system may differ, for other chemical synthesis systems.

FIG. 2A further illustrates standby line 148, standby circulator 150, and valves 149a, 149b, 149c, and 149d. These listed components provide the ability to recirculate heated loop gas back into the reactor rather than downstream for further processing. In other words, they provide a hot standby loop. In the case where feedstock, reactant, or other limited components may hinder methanol synthesis, a plant or system may utilize at least standby line 148, standby circulator 150, and valves 149a, 149b, 149c, and 149d to maintain the temperature of reactor 102 for an extended duration, or at least until the limited components are no longer limiting. One skilled in the art will appreciate that a plant or system can use a selection of 2 valves or diverters in place of valves 149a, 149b, 149c, and 149d. In another implementation, depending on plant needs, a greater number of valves can be used instead of valves 149a, 149b, 149c, and 149d. Valves, in this case, can be understood to be at least any of the following: gate valve, globe valve, ball valve, butterfly valve, diaphragm valve, plug valve, needle valve, pressure relief valve, control valve, three-way valve, diverter valve, piston valve, angle valve, foot valve, choke valve, safety valve, isolation valve, flow control valve, stop valve, swing check valve, wafer check valve, lift check valve, hydraulic valve, pneumatic valve, modulating valve, or the like. Other valve types will be apparent to those of skill in the art, any of which may be used.

A plant can selectively operate valves 149a and 149b to first divert the loop gas flow from the downstream cooling and/or separation process and direct it instead through standby line 148 (closing valve 149a and opening valve 149b). Circulator 150 can then be used to ensure proper movement of the loop gas through standby line 148. A plant can selectively operate valves 149c and 149d to complete the closed hot standby loop, relative to reactor 102. This loop then would continuously cycle the loop gas through reactor 102 and standby line 148a, with standby heater 144 keeping the loop gas at a temperature sufficient to prevent damage to reactor 102 or the catalyst inside, holding this part of the system in “standby” for as long as needed. Standby heater 144, in some implementations, can keep the loop gas at temperatures of 100° C. to 230° C. In another embodiment, yet another valve can be placed at the feedstock entrance to the reactor to prevent any backflow during the hot standby operation mode.

Though FIG. 2A illustrates the placement of all valves 149a, 149b, 149c, and 149d, in at least one embodiment, not all valves 149a, 149b, 149c, and 149d are used and a similar standby effect can still occur. For example, in a first example, hot standby could occur without the inclusion of valve 149a. In this first example, though a small portion of loop gas may backflow during the start-up of circulator 150, once started, circulator 150 would effectively cause the loop gas to circulate through the desired hot standby pathway. Similarly, valve 149c may not be used in a second example in which isolation of the circulator 150 is not desired or where isolation of the circulator 150 is achieved in another way. In at least one embodiment, valve 149c may be positioned between the standby heater 144 and the reactor 102. Thus, hot standby can be accomplished using only valves 149b and 149d (a valve that is not specific to the hot standby loop and is included only when no hot standby loop is provided).

One skilled in the art would recognize, given the present disclosure, that in the selective operation of valves 149a, 149b, 149c, and 149d, circulator 150, and standby heater 144, the reactor can be maintained at a safe idle temperature without the need for purging. Should a plant or system become aware of a need to limit its operation, e.g., within 1 hour, the methanol synthesis reaction can be halted, and the reactor can remain idle for hours or days, in such a hot standby mode. Accordingly, once the limiting restraint is no longer applicable, the plant can go from standby to production, e.g., within 1 hour. Such ability to quickly go from normal production to no production (in a hot standby mode) within a very short timeframe is incredibly advantageous, as normal shut down and start up procedures often take a day or more. Decreasing such time to less than 5 hours, less than 4 hours, less than 3 hours, less than 2 hours, or no more than about 1 hour is greatly advantageous.

During normal operation then, once the loop gas passes through the heat exchanger 104 and the heat exchanger 106, the product gas can be sent downstream to air cooler 108, where air cooler 108 removes heat from the product gas, e.g., down to a minimum practical temperature achievable by air cooling. For example, the product gas can enter air cooler 108 at approximately 100-120° C. and be cooled down to 50-60° C. Heat exchanger 104 can cool the product gas from an initial temperature of about 200-280° C. to a final temperature of about 190-230° C. In at least one embodiment, an exemplary plant may omit air cooler 108. The product gas is cooled further, e.g., by condenser 110. Condenser 110 can further remove heat from the product gas as well as latent heat such that methanol is condensed into its liquid phase. Condenser 110 may use cold water to sufficiently cool the product gas and form crude liquid methanol product. Such cooling within condenser 110 may be sufficient to ensure that the condensed crude methanol does not evaporate inside the catch pot 112. Any remaining gaseous fraction can flow out from the catch pot 112 (e.g., through valve 147c) either to be mixed back into the feedstock or can be purged from the plant or system. The condensed crude methanol can then be stored or sent downstream to be separated or otherwise purified.

Catch pot bypass line 146 and valves 147a, 147b, and 147c can also be used to implement an embodiment of a hot standby operation mode. For at least any of the already described reasons, should production drop below 50%, 40%, 35%, 30%, 25%, 20%, 15%, 10%, 5% (or another threshold value) of a typical operating flowrate, or the reaction becomes otherwise limited such that the reactor would need to be shut down, an operator can selectively operate valves 147a, 147b, and 147c to implement a hot standby operation. In that case, rather than allowing product gas to flow into condenser 110 and ultimately catch pot 112, an operator can selectively operate valves 147a and 147b, diverting the product gas through catch pot bypass line 146. A plant can additionally selectively operate valve 147c to ensure no bypassed product gas can enter the catch pot. A plant can then send such product gas (e.g., as a loop gas) back into the other areas of the plant to be mixed with the feedstock, purged, or continuously cycled during the hot standby operation. For example, as illustrated, such product gas could be looped back to the circulator noted to be upstream from feedstock line 101, providing a similar hot standby loop to that provided by reactor 102, standby line 148a, and standby heater 144.

In another embodiment, not all valves 147a, 147b, and 147c are used or included in a system to effectively provide the desired hot standby operation mode. For example, the hot standby operation mode may be effectuated using only valve 147b.

One skilled in the art will recognize, given the present disclosure, the benefits of operating an ETL plant by implementing such a bypass. Rather than expending resources to condense the product gas in condenser 110, and worrying about any undesired evaporation inside the catch pot, nominal catch pot levels can be maintained by diverting the relatively warm product loop gas around the condenser and catch pot. The loop gas can then circulate similarly to how standby line 148 circulates the loop gas through the reactor (i.e., with the difference being that the circulation loop is larger). During the hot standby without the bypass being activated, the loop gas may need to be cooled to 30-50° C. to prevent the evaporation of the liquid in the catch pot 112 and then reheated back to at least 210° C. by the standby heater 144. Diverting the loop gas through the bypass line 146 and thus isolating the catch pot 112 allows the loop gas to cool down to the maximum allowed inlet temperature of the main circulator, which can be 90-150° C. depending on the circulator type, materials of construction, cooling system, etc. This allows for a decrease in the reheating temperature difference of the loop gas from 160-190° C. to 60-120° C., therefore decreasing the energy consumption during hot standby.

In this case, the product loop gas can be fed through feedstock line 101 using a circulator and compressor to ensure that the reactor's 102 temperature remains high enough to avoid any needed shut down and reactor purge. As with the above-described hot standby operation modes, a heater (similar to standby heater 144) may keep the product loop gas at an optimal temperature of about 210° C. to 230° C. A plant can optimize its saving on equipment capacity by ensuring the loop gas remains hot enough to keep the reactor on a hot standby while also not requiring more expensive circulators rated for higher temperatures. A plant may also halt, or otherwise limit the cooling imparted on the loop gas by air cooler 108 and heat exchanger 104 during this period.

Embodiments of the present disclosure may also prove useful to plants and systems that are integrated with renewable energy sources or participate in grid balancing. These plants can utilize the hot standby operation modes during a period of time where only backup power or generator power is available, as the overall energy requirements associated with maintaining a hot standby mode can be much lower than during normal production mode.

In a given implementation of the present disclosure, a plant can utilize both standby line 148a and bypass line 146 (and include the associated valves and/or circulators). A relatively large plant, e.g. a 500 kiloton (KT) plant (i.e., a plant that produces 500 kilotons of methanol per year), will realize significant benefits by implementing a hot standby system (capable of a hot standby mode), including standby line 148, circulator 150, and valves 149a, 149b, 149c, and 149d. The overhead cost of implementing the hot standby mode in such an embodiment is a higher capital expense due to the additional required parts. However, such is particularly beneficial because the plant may stop the operation of the main syngas circulator and compressor and still maintain the hot standby operation. Instead, the movement of loop gas is limited to a dedicated standby circulator 150. By employing a relatively small standby circulator compared to the main syngas circulator, the electrical consumption required to facilitate the movement of loop gas through reactor 102 can be significantly reduced. For example, the electrical consumption can be reduced by 50% to 90%, and in some cases, as much as 95%. Further energy savings can be achieved by bypassing heat exchangers 104, 106, air cooler 108, and condenser 110 through hot standby line 148, which may also significantly reduce the energy consumption of heater 144 during hot standby operation (e.g., an electrical consumption reduction of 50% to 90%). The reduction in energy consumption reduces energy costs during hot standby and extends the operating window of the plant to lower minimum available power levels, reducing the frequency of cold start-ups due to lack of available intermittent power and associated costs of cold start-up, or reduces the minimum secure base load required to keep the loop hot and avoid the shutdown of the plant.

Implementing an embodiment including a catch pot bypass line 146 as shown in FIG. 2A provides an alternative embodiment whereby a smaller plant, such as a 100 KT plant, can achieve similar hot standby operations, without the need for valves 149a-149d and circulator 150. With a lower overhead cost, limited to mainly piping and valves/diverters, a plant can provide for a hot standby operation mode with significantly lower cost for reheating the loop gas, saving on the operation cost and expense associated with reactor shut down and start up. In at least one embodiment, a plant can implement both hot standby line 148 and catch pot bypass line 146 into its process. While ideally, a plant would keep the standby operation times to a minimum, the present disclosure provides ways to limit the overall time and costs associated with unforeseen or necessary downtime.

FIG. 2B schematically illustrates a plant or system 152b including a hot standby system (capable of implementing a hot standby mode). As illustrated, system 152b includes a standby steam heat exchanger 142. Standby steam heat exchanger 142 can be implemented into the above-described steam generation loops, like steam generation loops 131a and 131b. A plant may benefit from using any included steam generation loop to provide heating in either the hot standby operation mode or during the start-up of reactor 102, as this can reduce or eliminate the need to import steam from an outside source, or it may reduce the electricity otherwise needed. In another embodiment, standby steam heat exchanger 142 can be fed with steam from a variety of sources. System 152b additionally illustrates an embodiment wherein no air cooler is used in the cooling process of the loop gas exiting the reactor.

FIG. 2C schematically illustrates another plant or system 152c incorporating a hot standby loop otherwise similar to that shown in FIG. 2A, but wherein no air cooler and no standby steam heat exchanger are present.

### Section 3—Energy Efficiency Replacement System

FIGS. 3A and 3B schematically illustrate distillation or purification loops 164a, 164b of an exemplary ETL plant or system wherein varied configurations of heat supply systems are installed to provide thermal transfer. FIG. 3A illustrates a heat supply system or loops 164a comprising reboiler 154, steam boiler 158, and pump 156. In an embodiment, steam boiler 158 can be powered by electricity, a fuel or the like. FIG. 3B illustrates a loop 164b where the reboiler 154 is replaced with electric heater 162 (e.g., an electric immersion heater) and where the steam boiler 158, pump 156, and associating vessels, piping, and equipment are omitted.

A typical steam system, such as the steam boiler 158 seen in FIG. 3A can have an efficiency of approximately 80% due to heat losses across the system. One skilled in the art will appreciate that the configuration in FIG. 3B, however, can offer an even higher system efficiency, e.g., greater than 85%, greater than 90%, greater than 95%, or even greater than 99% vs the 80% found in the steam system of FIG. 3A. This increased efficiency, once again, can lead to lower energy consumption (lower operating costs). The utilization of the electric heater 162 can also provide the benefit of a lower capital expenditure for a plant or system, as the system of FIG. 3B is simpler, with less components.

FIG. 3B illustrates a loop 164b wherein steam is not utilized as a heating medium. Rather than utilizing a conventional reboiler, pump and steam boiler, (e.g., 154, 156 and 158 of FIG. 3A), loop 164b uses an electric heater 162 (e.g., a reboiler). This embodiment may include electric resistive heating coils positioned directly in communication with the process fluid that is cycled back to column 124. In some instances, this process fluid has a high fraction of water, which, if overheated, could potentially lead to scaling and a reduction in effective heat transfer. Another possible problem is thermal decomposition of various organic compounds in the water bottoms associated with column 124. The presently disclosed embodiments mitigate such risks by specifying a relatively low maximum thermal flux of approximately 40-50 kW/m² to limit the surface metal temperature of the coils of electric heater 162. While this embodiment may increase the cost of the electric coils required for effective operation, it reduces the risk of overheating the contents of the electric heater 162 (e.g., immersion heater). Such an embodiment can be highly cost effective with lower maintenance costs due to use of fewer pieces of equipment when compared to a conventional steam or oil heating system.

In another embodiment, electric heater 162 can be an electric process heater incorporated into the working medium loop downstream from heat exchanger 104 and upstream from heat exchanger 126 (see FIGS. 1A-1B). In other words, electric heater 162 of the system shown in FIG. 3B may be positioned between heat exchanger 104 and heat exchanger 126 of the system shown in FIGS. 1A-1B. This would cause the working medium loop to be larger, as the volumetric flowrate of the now electric working medium loop would have to be approximately 150-160% of the previously described working medium loop (that seen in FIGS. 1A-1B, without electric heater 162) if the loop is to be operated in the same temperature window as the base case of FIGS. 1A-1B (i.e., approximately 150-200° C.). Such a system allows a plant to physically relocate the electric heater in case the presence of an electric heater is deemed a safety hazard, positioned in close proximity to the distillation system. Additionally, overheating of the electric coils in the electric heater 162 is minimized, as the electric coil(s) are not in direct contact with the contents of the distillation column (but a working fluid). This also means that thermal degradation of the working medium is not a significant risk.

In yet another embodiment similar to loop 164b, rather than employing an electric heater 162, a plant can utilize a distinct thermal oil loop comprising an electric heater, a pump, and a reboiler. This system would transfer heat using the reboiler as the reboiler would be in fluid communication with the distillation column. A plant would advantageously benefit from this embodiment as the hot oil piping length can be shorter than that of a steam system, as the electric heater, pump, and reboiler can be located closer together, than is the case with a steam system. Additionally, such an embodiment allows for relocation of the electric heater in a situation where the presence of an electric heater is deemed a safety hazard, e.g., where close to the distillation system. In this configuration, the risk or effect of overheating of the electric coils in the electric heater is minimized, as the electric coil is not in direct contact with the product stream that flows through the distillation column, meaning no thermal degradation of process fluids is anticipated.

A plant can implement elements of any of the above-described implementations of various hot standby modes, high-temperature heat pump integration loops, and/or other described features to improve overall system energy efficiency. In one such embodiment, a plant or system can implement a similar hot standby system, as illustrated in FIGS. 2A-2C, into a high-temperature heat pump integration loop, as illustrated in FIGS. 1A-1B. One skilled in the art will appreciate the significant energy savings and the overall heat requirements savings that a plant or system can have when employing such an implementation. Further, a plant or system can incorporate the embodiments of loops 164a and 164b, shown in FIGS. 3A-3B, to further increase efficiency or to benefit from the varied configurations based on the specific needs of the plant or system.

Each of Applicants previous patents and application noted below is herein incorporated by reference in its entirety: U.S. application Ser. No. 18/986,261 filed Dec. 18, 2024, titled METHANOL DISTILLATION SYSTEM WITH ENHANCEMENTS FOR IMPROVED EFFICIENCY; U.S. Pat. No. 12,151,193 titled SYSTEM FOR SEPARATING GAS; U.S. Pat. No. 12,070,741 titled LOAD-FOLLOWING REACTOR SYSTEM, ASSOCIATED FACILITIES, AND METHOD OF OPERATING THE SAME; U.S. Pat. No. 11,738,317 titled REACTOR FOR SYNTHESIZING METHANOL OR OTHER PRODUCTS; U.S. Publication No. 2023/0271152 titled REACTOR FOR SYNTHESIZING METHANOL OR OTHER PRODUCTS; U.S. Pat. No. 12,098,657 titled HEAT INTEGRATION; U.S. Application 63/822,401 filed Jun. 12, 2025, titled COUNTERFLOW AND CROSSFLOW CHEMICAL SYNTHESIS REACTORS WITH CIRCUMFERENTIAL CHAMBERS; and U.S. Application 63/895,177 filed Oct. 7, 2025, titled METHANOL TO SUSTAINABLE AVIATION FUEL TECHNOLOGIES.

The present invention may be embodied in other specific forms without departing from its spirit or essential characteristics. The described embodiments are to be considered in all respects only as illustrative and not restrictive. The present invention may be a combination of any of the above described embodiments. The scope of the invention is, therefore, indicated by the appended claims rather than by the foregoing description. All changes that come within the meaning and range of equivalency of the claims are to be embraced within their scope.

1. A hot standby system for use in a chemical synthesis plant, comprising:
a feed line carrying a feed;
a reactor configured for receiving the feed, converting the feed through reaction over a catalyst, so as to produce a product loop gas that is output downstream from the reactor;
a heater operatively connected to the feed line and positioned upstream from the reactor;
a hot standby loop configured to selectively alter a flow path of the product loop gas when a flow of feed is below a minimum threshold, the hot standby loop putting the reactor in a hot standby mode when selectively activated;
a product loop gas cooler, downstream from the reactor and downstream from the hot standby loop, the product loop gas cooler being configured for removing heat from the product loop gas when the hot standby loop is not selectively activated;
a condenser, downstream from the reactor and downstream from the hot standby loop, wherein latent heat is removed from the product loop gas so as to form a condensed product loop liquid; and
a catch pot, downstream from the condenser, wherein the condensed product loop liquid is caught and at least partially retained in the catch pot, and wherein any uncondensed product loop gas is removed from the catch pot.

2. The hot standby system as recited in claim 1, wherein the feed line comprises a main feed line and a top feed line.

3. The hot standby system as recited in claim 2, wherein the heater is a standby heater, positioned on the top feed line.

4. The hot standby system as recited in claim 2, further comprising an interchanger operatively connected to the main feed line and a heating medium, the interchanger being configured to transfer heat between feed in the main feed line and the heating medium.

5. The hot standby system as recited in claim 2, wherein the hot standby loop comprises:
a standby loop line, wherein the product loop gas can flow, fluidly connected to a loop gas output and downstream from the reactor;
a standby circulator configured to facilitate the continuous movement of the product loop gas within the hot standby loop;
a first valve operatively connected to the standby loop line upstream of the standby circulator configured to selectively impede the product loop gas from moving through the hot standby loop; and
a second valve operatively connected to the top feed line to selectively impede flow through the top feed line,
wherein the first and second valves operate to alter a flow path of the product loop gas,
wherein opening the first valve, closing the second valve and turning on the standby circulator causes the product loop gas to flow circularly through the reactor and the hot standby loop, keeping the reactor in the hot standby mode until the flow of feed surpasses the minimum threshold,
wherein closing the first valve and opening the second valve returns the reactor to a normal operating mode.

6. The hot standby system as recited in claim 2, wherein the hot standby loop comprises:
a standby loop line, wherein the product loop gas can flow, fluidly connected to a loop gas output and downstream from the reactor;
a first valve operatively connected to the loop gas output to impede the product loop gas flow inside the loop gas output;
a standby circulator configured to facilitate the continuous movement of the product loop gas within the hot standby loop;
a second valve operatively connected to the standby loop line upstream of the standby circulator configured to selectively impede the product loop gas from moving through the hot standby loop;
a third valve operatively connected to the standby loop line downstream from the standby circulator configured to selectively impede flow through the standby loop line; and
a fourth valve operatively connected to the top feed line to selectively impede flow through the top feed line;
wherein the first, second, third, and fourth valves operate to alter a flow path of the product loop gas,
wherein closing of the first valve, opening of the second valve, opening of the third valve, and closing of the fourth valve causes the product loop gas to flow circularly through the reactor and the hot standby loop, keeping the reactor in the hot standby mode until the flow of feed surpasses the minimum threshold,
wherein opening the first valve and closing the second valve returns the reactor to a normal operating mode.

7. The hot standby system as recited in claim 1, wherein the feed is syngas comprising hydrogen and carbon dioxide.

8. The hot standby system as recited in claim 1, further comprising a catch pot bypass comprising:
a catch pot bypass line wherein product loop gas can flow;
a first valve that selectively allows the product loop gas to flow through the catch pot bypass;
a second valve operatively connected to the catch pot bypass line, wherein the second valve impedes the product loop gas from flowing through the catch pot bypass; and
a third valve operatively connected to the catch pot and selectively operable for impeding the flow of product loop gas, wherein closing of the first valve, opening of the second valve, and closing of the third valve cause the product loop gas to bypass the catch pot.

9. The hot standby system as recited in claim 1, further comprising a catch pot bypass comprising:
a catch pot bypass line wherein product loop gas can flow;
a first valve that selectively allows the product loop gas to flow through the catch pot bypass; and
a second valve operatively connected to the catch pot bypass line, wherein the second valve impedes the product loop gas from flowing through the catch pot bypass, wherein closing of the first valve and opening of the second valve cause the product loop gas to bypass the catch pot.

10. A hot standby system for use in a chemical synthesis plant, comprising:
a feed line carrying a feed;
a reactor configured for receiving the feed, converting the feed through reaction over a catalyst, so as to produce a product loop gas that is output downstream from the reactor;
a heater operatively connected to the feed line and positioned upstream from the reactor;
a product loop gas cooler, downstream from the reactor, the product loop gas cooler being configured for removing heat from the product loop gas;
a condenser, downstream from the reactor and downstream from the hot standby loop, wherein latent heat is removed from the product loop gas so as to form a condensed product loop liquid;
a catch pot bypass configured to selectively alter a flow path of the product loop gas when a flow of feed is below a minimum threshold, the catch pot bypass loop putting the reactor in a hot standby mode when selectively activated; and
a catch pot, downstream from the condenser, wherein during a normal operation mode when the flow of feed is above the minimum threshold, the condensed product loop liquid is caught and at least partially retained in the catch pot, and wherein any uncondensed product loop gas is removed from the catch pot;
wherein when the reactor is in the hot standby mode and the catch pot bypass is activated, the catch pot is bypassed.

11. The standby system as recited in claim 10, wherein the feed line further comprises a main feed line and a top feed line.

12. The standby system as recited in claim 11, further comprising an interchanger operatively connected to the main feed line and a heating medium, configured to transfer heat between the main feed and the heating medium.

13. The standby system as recited in claim 10, wherein the catch pot bypass comprises:
a catch pot bypass line wherein product loop gas can flow;
a first valve operatively connected to the reactor and selectively allows the product to flow through the catch pot bypass;
a second valve operatively connected to the catch pot bypass line wherein the second valve impedes the product loop gas from flowing through the catch pot bypass; and
a third valve operatively connected to the catch pot and selectively operable for impeding the flow of product loop gas.

14.-16. (canceled)

17. A method of operating a chemical synthesis plant in a hot standby mode, the method comprising:
reducing a feed flow rate;
selectively actuating at least one valve, wherein the actuating of the at least one valve is configured to direct a product loop gas from a standard flow path to a standby flow path, and
wherein the standby flow path is a closed loop; and
circulating the product loop gas within the standby flow path for a duration, until the reduced feed flow rate increases to a given threshold value.

18. The method as recited in claim 17, further comprising heating the product loop gas with an electric heater within the standby flow path.

19. The method as recited in claim 17, further comprising circulating the product loop gas within the standby flow path through a fluidly connected reactor,
wherein the product loop gas maintains the reactor within a specified temperature range to prevent a cold shutdown of such reactor.

20. The method as recited in claim 17, wherein one or more valves are selectively actuated to alter the standard flow path and form the standby flow path.

21. The method as recited in claim 17, wherein the reduced feed flow rate threshold value is less than or equal to about 30% of a working flow rate associated with the standard flow path.

22. The method as recited in claim 17, wherein the chemical synthesis plant comprises a methanol synthesis plant.

23. The method as recited in claim 17, further comprising selectively actuating at least one valve once the reduced feed flow rate increases by the given threshold value,
wherein the actuating of the at least one valve is configured to direct the product loop gas from the standby flow path to the standard flow path.

24.-25. (canceled)
